(19) United States
(12) Patent Application Publication
(10) Pub. No.: US 2026/0337074 A1
(43) Pub. Date: Oct. 29, 2026

(54) MODIFIED-ZEOLITE IMMOBILIZED ALGAE BEADS, PREPARATION METHOD AND USE THEREOF

(71) Applicant: GUANGDONG SHUNKONG ZIHUA TECHNOLOGY CO., LTD., Foshan (CN)

(72) Inventors: Yan WANG, Foshan (CN); Bin ZHU, Foshan (CN); Jinquan WAN, Foshan (CN); Xuejian WANG, Foshan (CN); Shiming JI, Foshan (CN)

(21) Appl. No.: 18/841,880

(22) PCT Filed: Dec. 22, 2023
(86) PCT No.: PCT/CN2023/141134

§ 371 Date: Aug. 27, 2024

(30) Foreign Application Priority Data
Dec. 4, 2023 (CN) 202311653214.4

(51) Int. Cl. C02F 3/32; C01B 39/02; C02F 3/00

(52) U.S. Cl. CPC C02F 3/322; C01B 39/026; C02F 2003/001

(57) ABSTRACT

The present application relates to modified-zeolite immobilized algae beads and a preparation method thereof and use thereof. The modified-zeolite immobilized algae beads are prepared by modifying zeolite with sodium bicarbonate and chitosan, followed by immobilizing the modified zeolite with Chlorella in embedding gel beads to achieve the immobilization of the algae beads.

### CROSS-REFERENCE TO RELATED APPLICATIONS

This application is an U.S. national phase application under 35 U.S.C. § 371 based upon international patent application No. PCT/CN2023/141134 filed on Dec. 22, 2023, which itself claims priority to Chinese patent application No. 202311653214.4, titled “MODIFIED-ZEOLITE IMMOBILIZED ALGAE BEADS, PREPARATION METHOD AND USE THEREOF”, filed on Dec. 4, 2023. The contents of the above-identified applications are hereby incorporated herein in their entireties by reference.

### TECHNICAL FIELD

The present application relates to the field of wastewater treatment technology, particularly relating to modified-zeolite immobilized algae beads, a preparation method thereof, and use thereof.

### BACKGROUND

With the rapid growth of the economy and the ongoing improvement of people's living standards, there is an increasing demand for the quality of livestock and poultry products. This demand further promotes the high-quality development of related aquaculture industries. The tailwater from aquaculture bases after high-density breeding contains a significant amount of pollutants such as NH₄⁺-N, organic matter, and NO₃⁻-N. As a result, untreated tailwater discharge could deteriorate the water quality of local areas and reduce the concentration of dissolved oxygen in the water.

Studies have shown that Chlorella is highly effective at removing certain pollutants such as nitrogen and phosphorus from water. Chlorella is able to synthesize cellular components and release oxygen through photosynthesis using the nutrients in wastewater. However, due to the small size of Chlorella, using it directly to treat wastewater faces challenges such as poor impact resistance, easy loss, and difficulty in harvesting, failing to meet water treatment standards. Chlorella can be encapsulated in gel beads by immobilization technology, which, however, limits its mass transfer performance, reducing the degradation efficiency of nitrogen and phosphorus pollutants in water. Moreover, after long-term use, some algal cells may leak from the gel beads, causing significant toxicity to microorganisms.

Zeolite possesses a large specific surface area, strong electrostatic and dispersion forces, and robust adsorption capability. Additionally, the lattice of zeolite contains ions such as K⁺, Na⁺, and Ca²⁺, which can exchange with other cations in solution without compromising the stability of the zeolite structure. Due to the adsorption and cation exchange capabilities, zeolite has excellent adsorption effect on NH₄⁺-N. To maximize the adsorption effect, zeolite is often crushed to micron sizes in practical use. However, the small diameters make it difficult for zeolite to settle quickly and separate from wastewater, leading to increased processing time and cost. Moreover, zeolite has relatively low adsorption capacity for NO₃⁻-N in wastewater, and requires further development for removing total phosphorus (TP), chemical oxygen demand (COD), and total nitrogen (TN).

### SUMMARY

According to various embodiments of the present application, modified-zeolite immobilized algae beads and a preparation method thereof and use thereof are provided.

In a first aspect of the present application, a method for preparing modified-zeolite immobilized algae beads is provided, including the following steps:
obtaining porous zeolite;
mixing the porous zeolite with a mixed solution including chitosan, glacial acetic acid, and water to prepare chitosan-modified porous zeolite; and
mixing the chitosan-modified porous zeolite with an embedding agent, Chlorella, and a cross-linking agent to prepare the modified-zeolite immobilized algae beads.

In an embodiment, the chitosan includes at least one of the following characteristics:
(1) deacetylation degree ≥95%;
(2) viscosity is ranged from 100 mPa·s to 200 mPa·s.

In an embodiment, the preparation of the chitosan-modified porous zeolite satisfies at least one of the following conditions:
(1) the mass fraction of chitosan in the mixed solution is ranged from 0.4% to 1.5%;
(2) the mass fraction of glacial acetic acid in the mixed solution is ranged from 2% to 6%;
(3) the mass to volume ratio of porous zeolite to the mixed solution is ranged from (2 to 6) g:100 mL.

In an embodiment, the embedding agent includes polyvinyl alcohol and sodium alginate.

In an embodiment, the cross-linking agent includes a saturated boric acid solution and calcium chloride.

In an embodiment, the mass ratio of polyvinyl alcohol to sodium alginate is ranged from (4 to 8):(2 to 4); the mass ratio of polyvinyl alcohol to the chitosan-modified porous zeolite is ranged from (4 to 8):(2 to 6).

In an embodiment, the mass to volume ratio of calcium chloride to the saturated boric acid solution is ranged from (10 to 20) g:500 mL.

In an embodiment, the porous zeolite includes Na-type porous zeolite.

In an embodiment, a preparation method for the Na-type porous zeolite includes the following steps:

adding zeolite to a sodium bicarbonate solution, and heating at a temperature ranged from 300° C to 450° C, thereby forming the Na-type porous activated zeolite.

In an embodiment, the preparation of the Na-type porous zeolite satisfies at least one of the following conditions:
(1) the zeolite includes one or more of clinoptilolite, mordenite, heulandite, or chabazite;
(2) the concentration of the sodium bicarbonate solution is ranged from 0.7 mol/L to 1.0 mol/L;
(3) the mass to volume ratio of the zeolite to the sodium bicarbonate solution is ranged from (10 to 30) g:1 L;
(4) the duration of heating is ranged from 120 min to 240 min.

In an embodiment, the diameter of the modified-zeolite immobilized algae beads is ranged from 4.2 mm to 5.4 mm.

In an embodiment, the density of the modified-zeolite immobilized algae beads is ranged from 1.09 g/cm³ to 1.21 g/cm³.

In a second aspect of the present application, modified-zeolite immobilized algae beads are provided, which are prepared using the method described in the first aspect.

In a third aspect of the present application, use of the modified-zeolite immobilized algae beads described in the second aspect in wastewater treatment is provided.

In an embodiment, the use includes introducing the modified-zeolite immobilized algae beads at a ratio ranged from 10 g/L to 20 g/L into wastewater for water treatment, wherein the C/N ratio of the wastewater is ranged from 1 to 3.

Details of one or more embodiments of the present application are set forth in the following description, and other features, objectives, and advantages of the present application will become apparent from the specification and claims.

### DETAILED DESCRIPTION

The technical solutions in embodiments of the present application will now be clearly and fully described in detail with reference to the embodiments. The embodiments described herein are only some but not all embodiments of the present application. Based upon the embodiments of the present application, all other embodiments obtained by those of ordinary skill in the art without creative efforts should fall within the scope of protection of the present application.

Unless defined otherwise, all technical and scientific terms used herein have the same meaning as commonly understood by those skilled in the art of this application. The terms used in the specification of the present application are for the purpose of describing exemplary examples only and are not intended to limit the present application.

Unless otherwise stated or contradicted, the terms or phrases used herein have the following meanings:

In the present application, “one or more” refers to any one, any two, or more than two of the listed items.

In the present application, the terms “and/or”, “or/and”, “as well as/or” when used in a list of two or more associated items, mean that any one of the items can be selected, and any or all combinations of the items can be selected. The any or all combinations include the combination of any two of the associated listed items, the combination of any more of the associated listed items, or the combination of all associated listed items.

In the present application, “further”, “furthermore”, “particularly”, etc., are used for descriptive purposes, indicating differences in content, but should not be construed as limiting the protection scope of the present application.

In the present application, “first aspect,” “second aspect,” “third aspect,” “fourth aspect,” etc., are used only for descriptive purposes and should not be understood as indicating or implying relative importance or quantity, nor should they be understood as implicitly indicating the importance or quantity of the technical features referred to. Furthermore, “first,” “second,” “third,” “fourth,” etc., are used for non-exhaustive descriptive purposes, not constituting a closed-end limitation on quantity. Accordingly, features modified with “first,” “second,” etc., can explicitly or implicitly include at least one such features. In the description of the present application, “multiple” means at least two, such as two, three, etc., unless otherwise specifically defined. In the description of the present application, “several” means at least one, such as one, two, etc., unless otherwise specifically defined.

In the present application, unless otherwise specified, when a numerical interval is referred to, the numerical interval is continuous and include the minimum value and the maximum value of the range, as well as every value between the minimum value and the maximum value. When a numerical interval is referred to as encompassing only integers, it includes every integer between the minimum value and the maximum value. Furthermore, when multiple ranges are provided to describe a feature or a characteristic, these ranges can be combined. In other words, ranges disclosed herein are to be construed to include any or all sub-ranges subsumed therein, unless otherwise specified.

In the present application, unless specifically indicated otherwise, for solid-liquid mixtures and solid-solid mixtures, the percent contents refer to the mass percentages, and for liquid-liquid mixtures, the percent contents refer to the volume percentages.

In the present application, unless specifically stated, the percent concentrations refer to the final concentrations. The final concentration refers to the proportion of an added component in the system after the addition of that component.

In the present application, unless specifically limited, the temperature parameters allow for both constant temperature treatment and treatment within a certain temperature range. The constant temperature treatment allows for fluctuations within the precision range of the instrument, such as ±5° C, ±2° C, ±1° C, ±0.5° C, ±0.4° C, ±0.3° C, ±0.2° C, ±0.1 °C In the present application, room temperature refers to the condition without temperature control, generally is 4° C to 35° C, preferably about 20±5 °C

In the present application, when describing technical features in an open-ended way, the scope encompasses both a closed-ended technical solution consisting of the listed features and an open-ended technical solution including the listed features.

Natural zeolite is a widely distributed silicate mineral material, composed of silicon-oxygen and aluminum-oxygen tetrahedral three-dimensional frameworks interconnected by shared oxygen atoms. As a non-metallic mineral material, natural zeolite has many advantages including abundant reserves and low costs, and exhibits properties such as adsorption, ion exchange, and shape-selective catalysis. Particularly, natural zeolite has high selective adsorption of pollutants such as ammonium and nitrogen, and thus is commonly used as an ammonium nitrogen adsorbent. In practical use, natural zeolite is often crushed to micron sizes to increase its specific surface area and maximize its adsorption effect, thereby enhancing its ammonium nitrogen removal efficiency. However, when crushed zeolite is introduced in wastewater for adsorption, the small diameters make it difficult for crushed zeolite to settle quickly and effectively separate from wastewater.

Moreover, although zeolite has good adsorption properties, particularly excellent adsorption capacity for NH₄⁺-N in wastewater, its adsorption capacity for NO₃⁻-N in water is relatively poor. Chitosan can adsorb NO₃⁻-N in water and, through adsorption and net trapping effects, promote algae cell flocculation and concentration, reducing algae cell loss. However, when used alone, chitosan is unstable and is prone to aggregation, affecting the effectiveness of wastewater treatment.

Immobilized microorganism technology is a biotechnological technology that involves fixing selected microorganisms onto a specific carrier, allowing the microorganisms to remain highly dense and maintain biological activity, and under suitable conditions, allowing the microorganisms to rapidly and extensively proliferate. Applying such technology in wastewater treatment is beneficial to increasing the concentration of microorganisms (especially those with special functions) in a bioreactor, helps the microorganisms resisting adverse environmental impacts, facilitates the solid-liquid separation after the reaction, and shortens the required treatment time. Embedding is the most common method for immobilizing microorganisms. The principle of embedding involves trapping microorganism cells in the network of pores of water-insoluble gel polymer, and retaining cells through polymerization, ion network formation, or precipitation, or by altering solvent, temperature, or pH value. The network of the gel polymer can prevent cell leakage while allowing medium to penetrate and products to diffuse out.

The technicians of the present application unexpectedly discovered in experiments that porous activated zeolite can be introduced into a solution of chitosan and glacial acetic acid, followed by stirring to obtain chitosan-modified porous zeolite. The chitosan-modified porous zeolite can be mixed with an embedding agent uniformly, and added with a cultured Chlorella algal solution to form an algal mixture. The algal mixture can be added with a cross-linking agent to obtain modified-zeolite immobilized algae beads. Immobilizing the modified zeolite and Chlorella in embedding gel beads not only enhances the removal effects on pollutants in water but also minimizes microbial toxicity and makes it easier to separate Chlorella and zeolite from wastewater.

In a first aspect of the present application, a method for preparing modified-zeolite immobilized algae beads is provided, including the following steps:
obtaining porous zeolite;
mixing the porous zeolite with a mixed solution including chitosan, glacial acetic acid, and water to prepare chitosan-modified porous zeolite; and
mixing the chitosan-modified porous zeolite with an embedding agent, Chlorella, and a cross-linking agent to prepare the modified-zeolite immobilized algae beads.

The present application employs chitosan to modify zeolite, which enhances the adsorption capacity of zeolite for NH₄⁺—N and enables the zeolite to adsorb NO₃⁻—N.

Subsequently, the chitosan-modified zeolite and Chlorella are immobilized in embedding gel beads, which not only improves the removal effects on pollutants in water, and but also reduces the leakage and loss of algal cells from the gel beads, thereby minimizing microbial toxicity.

Furthermore, the prepared modified-zeolite immobilized algae beads exhibit good stability, appropriate diameters and densities, and significantly improved removal effects on NH₄⁺—N, NO₃⁻—N, TP, COD, and TN in wastewater. The mechanical strength, embedding performance, and service life of the algae beads can be enhanced. Additionally, the operational difficulty and cost of separating Chlorella and zeolite from wastewater can be reduced, demonstrating promising application prospects.

Specifically, the amino and hydroxyl groups in chitosan undergo protonation, forming ion pairs with NO₃⁻—N in water through electrostatic adsorption. Since zeolite has a high surface area and strong mechanical stability, incorporating zeolite into chitosan enhances mechanical properties of chitosan. The zeolite adhered with chitosan effectively adsorbs NO₃⁻—N in water. Meanwhile, chitosan can promote algae cell flocculation and concentration through adsorption, electro-neutralization, and high molecular weight net trapping effects, reducing the loss and leakage of algae cells.

In some embodiments, the degree of deacetylation of chitosan is greater than or equal to 95%. It can be understood that the degree of deacetylation of chitosan includes, but is not limited to, 95%, 96%, 97%, 98%, and 99%.

In some embodiments, the viscosity of the chitosan is 100 m·Pas to 200 mPa·s. It can be understood that the viscosity of chitosan includes, but is not limited to, 100 m·Pas, 110 m·Pas, 120 m·Pas, 130 m·Pas, 140 m·Pas, 150 m·Pas, 160 m·Pas, 170 m·Pas, 180 m·Pas, 190 mPa·s, and 200 m·Pas.

In some embodiments, during the preparation of the chitosan-modified porous zeolite, the mass fraction of chitosan in the mixed solution is 0.4% to 1.5%. It can be understood that the mass fraction of chitosan in the mixed solution includes, but is not limited to, 0.4%, 0.5%, 0.6%, 0.7%, 0.8%, 0.9%, 1.0%, 1.1%, 1.2%, 1.3%, 1.4%, and 1.5%.

Furthermore, the zeolite adhered with chitosan can effectively adsorb NO₃⁻—N in water, and chitosan can promote algae cell flocculation and concentration through adsorption, electro-neutralization, and high molecular weight net trapping effects, reducing the loss and leaching of algae cells. However, an over high concentration of chitosan solution can reduce the adsorption performance of zeolite for NH₄⁺—N.

In some embodiments, during the preparation of the chitosan-modified porous zeolite, the mass fraction of glacial acetic acid in the mixed solution is 2% to 6%. It can be understood that the mass fraction of glacial acetic acid in the mixed solution includes, but is not limited to, 2%, 3%, 4%, 5%, and 6%.

In some embodiments, during the preparation of the chitosan-modified porous zeolite, the mass to volume ratio of the porous zeolite to the mixed solution is (2 to 6) g:100 mL. It can be understood that the mass to volume ratio of the porous zeolite to the mixed solution includes, but is not limited to, 2 g:100 mL, 3 g:100 mL, 4 g:100 mL, 5 g:100 mL, and 6 g:100 mL.

In some embodiments, the embedding agent includes polyvinyl alcohol and sodium alginate. The addition of polyvinyl alcohol enhances the strength of the gel beads. By using sodium alginate and polyvinyl alcohol as the embedding agent, a gel with a porous network structure can be formed. By incorporating zeolite into the embedding agent, the mechanical strength of the sodium alginate/polyvinyl alcohol gel beads can be increased, the adsorption capacity for TN in water can be enhanced, the algal biofilm formed by Chlorella is not easily to lose, and zeolite can achieve biological regeneration through Chlorella.

In some embodiments, the mass ratio of polyvinyl alcohol to sodium alginate is (4 to 8):(2 to 4). It can be understood that the mass ratio of polyvinyl alcohol to sodium alginate includes, but is not limited to, 4:2, 4:3, 4:4, 5:2, 5:3, 5:4, 6:2, 6:4, 7:2, 7:3, 7:4, 8:2, and 8:3.

In some embodiments, the mass ratio of polyvinyl alcohol to the chitosan-modified porous zeolite is (4 to 8):(2 to 6). It can be understood that the mass ratio of polyvinyl alcohol to the chitosan-modified porous zeolite includes, but is not limited to, 4:2, 4:3, 4:4, 4:5, 4:6, 5:2, 5:3, 5:4, 5:6, 6:2, 6:4, 6:5, 7:2, 7:3, 7:4, 7:5, 7:6, 8:2, 8:3, 8:5, and 8:7.

Furthermore, the addition of polyvinyl alcohol is beneficial for enhancing the strength of the gel beads, but an over high concentration of polyvinyl alcohol can reduce the mass transfer properties of the gel beads.

In a specific embodiment, the polyvinyl alcohol includes one or more of polyvinyl alcohol 124, polyvinyl alcohol 1788, polyvinyl alcohol 1799, or polyvinyl alcohol 2488.

In a specific embodiment, the degree of alcoholysis of the polyvinyl alcohol is 98% to 99%. It can be understood that the degree of alcoholysis of the polyvinyl alcohol includes, but is not limited to, 98%, 98.2%, 98.4%, 98.6%, 98.8%, and 99%.

In a specific embodiment, the average degree of polymerization of the polyvinyl alcohol is 2400 to 2500. It can be understood that the average degree of polymerization of the polyvinyl alcohol include, but is not limited to, 2400, 2410, 2420, 2430, 2440, 2450, 2460, 2470, 2480, 2490, and 2500.

In a specific embodiment, the viscosity of the polyvinyl alcohol is 54 mPa·s to 66 mPa·s. It can be understood that the viscosity of the polyvinyl alcohol includes, but is not limited to, 54 m·Pas, 55 m·Pas, 56 mPa s, 57 m·Pas, 58 m·Pas, 59 mPa s, 60 m·Pas, 61 mPa s, 62 mPa s, 63 m·Pas, 64 m·Pas, 65 m·Pas, and 66 mPa s.

In some embodiments, the cross-linking agent includes a saturated boric acid solution and calcium chloride.

Furthermore, the cross-linking agent includes both boric acid and calcium chloride. Calcium chloride reacts with sodium alginate to form stable gel beads, while boric acid cross-links with polyvinyl alcohol to form stable acetal condensates. This also facilitates ionic exchange between calcium chloride and sodium alginate, further enhancing the mechanical properties of the immobilized algae beads.

In some embodiments, during the preparation reaction of the cross-linking agent, the mass to volume ratio of calcium chloride to the saturated boric acid solution is (10-20) g:500 mL. It can be understood that the mass to volume ratio of calcium chloride to the saturated boric acid solution includes, but is not limited to, 10 g:500 mL, 12 g:500 mL, 14 g:500 mL, 16 g:500 mL, 18 g:500 mL, and 20 g:500 mL.

In some embodiments, the porous zeolite includes Na-type porous zeolite.

In some embodiments, a preparation method for the Na-type porous zeolite includes the following steps:
adding zeolite to a sodium bicarbonate solution, and heating at 300° C to 450° C, thereby forming the Na-type porous activated zeolite.

Furthermore, during the heating process of the zeolite, the sodium bicarbonate immersed within the structure of the zeolite decomposes into CO₂ and H₂O at the high temperature, creating more pores, and increasing the average pore size of the zeolite, resulting in an increased surface area. Additionally, the Na⁺ doping enhances the ion exchange capability of the zeolite with NH₄⁺—N in water, providing more active sites for NH₄⁺—N adsorption.

In some embodiments, in the preparation of the Na-type porous zeolite, the zeolite includes one or more of clinoptilolite, mordenite, heulandite, or chabazite.

In some embodiments, the particle size of the zeolite is 0.3 mm to 0.5 mm. It can be understood that the particle size of the zeolite includes, but is not limited to, 0.3 mm, 0.35 mm, 0.4 mm, 0.45 mm, and 0.5 mm.

In some embodiments, in the preparation of the Na-type porous zeolite, the concentration of the sodium bicarbonate solution is 0.7 mol/L to 1.0 mol/L. It can be understood that the concentration of the sodium bicarbonate solution includes, but is not limited to, 0.7 mol/L, 0.8 mol/L, 0.9 mol/L, and 1.0 mol/L.

In some embodiments, in the preparation of the Na-type porous zeolite, the mass to volume ratio of the zeolite to the sodium bicarbonate solution is (10 to 30) g:1 L. It can be understood the mass to volume ratio of the zeolite to the sodium bicarbonate solution includes, but is not limited to, 10 g:1 L, 15 g:1 L, 20 g:1 L, 25 g:1 L, and 30 g:1 L.

In some embodiments, in the preparation of the Na-type porous zeolite, the duration of heating is 120 min to 240 min. It can be understood that the duration of heating includes, but is not limited to, 120 min, 140 min, 160 min, 180 min, 200 min, 220 min, and 240 min.

In some embodiments, in the preparation of the Na-type porous zeolite, the equipment used for the high-temperature heating is a muffle furnace; the heating rate of the muffle furnace is 4° C/min to 6° C/min. It can be understood that the heating rate of the muffle furnace includes, but is not limited to, 4° C/min, 5° C/min, and 6° C/min.

In some embodiments, the diameters of the modified-zeolite immobilized algae beads are 4.2 mm to 5.4 mm. It can be understood that the diameters of the modified-zeolite immobilized algae beads include, but are not limited to, 4.2 mm, 4.4 mm, 4.6 mm, 4.8 mm, 5.0 mm, 5.2 mm, and 5.4 mm.

In some embodiments, the density of the modified-zeolite immobilized algae beads is 1.09 g/cm³ to 1.21 g/cm³. It can be understood that the density of the modified-zeolite immobilized algae beads includes, but is not limited to, 1.09 g/cm³, 1.11 g/cm³, 1.13 g/cm³, 1.15 g/cm³, 1.17 g/cm³, 1.19 g/cm³, and 1.21 g/cm³.

In some embodiments, the method for preparing the modified-zeolite immobilized algae beads includes the following steps:
(1) introducing zeolite to a sodium bicarbonate solution, stirring, filtering, drying, and then heating at a temperature of 300° C to 450° C, thereby forming Na-type porous activated zeolite;
(2) mixing water, chitosan, and glacial acetic acid to prepare a first mixture; adding the Na-type porous activated zeolite to the first mixture, followed by stirring, filtering, drying, and grinding, thereby forming Na—CS porous activated zeolite;
(3) mixing polyvinyl alcohol, sodium alginate, the Na—CS porous activated zeolite, and water, and stirring to prepare a second mixture;
(4) sterilizing the second mixture with high-temperature steam, cooling to room temperature, and then mixing with Chlorella, and stirring to prepare a third mixture;
(5) adding calcium chloride to a saturated boric acid solution and adjusting the pH value of the resulting solution to 6.4 to 6.6 with sodium bicarbonate, followed by high-temperature steam sterilization to prepare a cross-linking agent; dropping the third mixture into the cross-linking agent, and performing low-temperature immobilization cross-linking, thereby forming the modified-zeolite immobilized algae beads.

In some embodiments, in step (3), the mass to volume ratio of the Na—CS porous activated zeolite to water is (2 to 6) g:100 mL. It can be understood that the mass to volume ratio of the Na—CS porous activated zeolite to water includes, but is not limited to, 2 g:100 mL, 3 g:100 mL, 4 g:100 mL, 5 g:100 mL, and 6 g:100 mL.

In some embodiments, in step (3), the mass to volume ratio of polyvinyl alcohol to water is (4 to 8) g:100 mL. It can be understood that the mass to volume ratio of polyvinyl alcohol to water includes, but is not limited to, 4 g:100 mL, 5 g:100 mL, 6 g:100 mL, 7 g:100 mL, and 8 g:100 mL.

In some embodiments, in step (3), the mass to volume ratio of sodium alginate to water is (2 to 4) g:100 mL. It can be understood that the mass to volume ratio of sodium alginate to water includes, but is not limited to, 2 g:100 mL, 3 g:100 mL, and 4 g:100 mL.

In some embodiments, step (5) includes: dropping the third mixture into the prepared cross-linking agent drop by drop using a 5 ml syringe to form immobilized algae beads, which are then transferred to a refrigerator and stored at 4° C for low-temperature immobilization cross-linking for 24 hours.

In some embodiments, the syringe needle drops the third mixture into the cross-linking agent from a distance of 10 cm to 15 cm, at a rate of 1 drop per second, and the operation is completed at a temperature of 25° C to 30 °C

In a second aspect of the present application, modified-zeolite immobilized algae beads are provided, which are prepared using the method described in the first aspect.

The present application employs chitosan to modify zeolite, which enhances the adsorption capacity of zeolite for NH₄⁺—N and enables the zeolite to adsorb NO₃⁻—N. Subsequently, the chitosan-modified zeolite and Chlorella are immobilized in embedding gel beads, which improves the removal effects on pollutants in water, reduces the leakage and loss of algal cells from the gel beads, and makes it easier to separate Chlorella and zeolite from wastewater.

In a third aspect of the present application, use of the modified-zeolite immobilized algae beads described in the second aspect in wastewater treatment is provided.

In some embodiments, the C/N ratio of the wastewater is ranged from 1 to 3.

In some embodiments, the use includes introducing the modified-zeolite immobilized algae beads at a ratio of 10 g/L to 20 g/L into wastewater for the treatment.

In some embodiments, the use includes introducing the modified-zeolite immobilized algae beads at a ratio of 10 g/L to 20 g/L into wastewater for the treatment, wherein the C/N ratio of the wastewater is ranged from 1 to 3.

In some embodiments, after the wastewater treatment steps described above, in the treated wastewater, the removal rate of NH₄⁺—N is above 75%, the removal rate of NO₃⁻—N is above 55%, the removal rate of TP is above 61%, the removal rate of TN is above 58%, and the removal rate of COD is above 60%.

The following further elaborates in combination with specific examples. Unless specially stated, all the raw materials involved in the following specific examples can be sourced from commercial suppliers; the instruments used, unless specifically stated, can also be obtained from commercial sources; and the processes involved, unless specifically stated, are those commonly adopted by the skilled in the art. These processes can refer to the guidance provided in the present application, experimental manuals in the field, other known experimental methods in the field, or experimental conditions recommended by manufacturers.

The parameters and preparation methods of raw materials used in examples and comparative examples of the present application are as follows:

The zeolite raw material was natural clinoptilolite, purchased from Henan Guanghui Water Treatment Material Co., Ltd.

Chitosan with a deacetylation degree of 95.0% and a viscosity of 105 mPa·s can be purchased from Shanghai Macklin Biochemical Co., Ltd., under the model number C804726-100 g.

Glacial acetic acid with a concentration of 99.5 wt % can be purchased from Shanghai Macklin Biochemical Co., Ltd., under the model number A801295-500 ml.

Polyvinyl alcohol was polyvinyl alcohol 124 with an alcoholysis degree of 99.0%, an average degree of polymerization of 2500, and a viscosity of 56 mPa·s, which can be purchased from Shanghai Macklin Biochemical Co., Ltd., under the model number P909857-100 g.

Sodium alginate can be purchased from Shanghai Macklin Biochemical Co., Ltd., under the model number S817374-100 g.

Boric acid can be purchased from Shanghai Macklin Biochemical Co., Ltd., under the model number B802844-500 g.

BG-11 culture medium can be purchased from Qingdao Hi-Tech Industrial Park Haibo Biotechnology Co., Ltd., under the model number HB8793.

Method for preparing the Chlorella algae solution: Chlorella was inoculated into the BG-11 culture medium and cultured for 7 days under the culture conditions: temperature 25° C to 30° C, illumination 5000±1000 Lux, and pH value adjusted to 6 to 7 with sodium bicarbonate, to prepare the Chlorella algae solution.

Method for preparing the saturated boric acid solution: Boric acid and water at a mass to volume ratio of 7 g:100 ml were mixed at room temperature, heated to 80° C to 100° C, and stirred thoroughly until all boric acid powder was dissolved. After natural cooling, the clear solution from the upper layer was taken as the saturated boric acid solution.

Method for determining the diameters of the immobilized algae beads: Ten immobilized algae beads of uniform shape and size were taken and the diameters thereof were then measured using a vernier caliper. The average diameter was calculated from these measurements.

Method for determining the density of the immobilized algae beads: Ten immobilized algae beads were weighed and the masses thereof were recorded, and then placed in a graduated cylinder filled with a predetermined volume of water. The volume changes in the graduated cylinder before and after adding the immobilized algae beads were recorded. The density of the immobilized algae beads can be calculated using the density formula.

### Example 1

(1) The zeolite raw material was washed with deionized water three times, dried in a 60° C oven for 240 minutes. Then the natural zeolite was added to 0.7 mol/L sodium bicarbonate solution at a mass to volume ratio (g/L) of 10:1, stirred at 200 r/min for 12 hours, filtered, and dried. The dried zeolite was heated in a muffle furnace at a heating rate of 4° C/min to 300° C and heated at this temperature for 120 minutes, to obtain Na-type porous activated zeolite.
(2) Chitosan was dissolved in water, stirred at 30° C for 5 minutes, then added with glacial acetic acid and mixed evenly to prepare a mixed solution including 0.4% chitosan and 4% glacial acetic acid. 2 g of the Na-type porous activated zeolite prepared in step (1) was added to 100 mL of the mixed solution, vibrated and stirred at 140 r/min for 24 hours, then washed with deionized water until the filtrate is neutral, dried at 60° C for 120 minutes to obtain Na—CS porous activated zeolite. The obtained Na—CS porous activated zeolite was ground and sieved through a 300-mesh screen to obtain uniformly sized Na—CS porous activated zeolite.
(3) 4 g of polyvinyl alcohol 124 was added to 100 ml of water, heated on an electric heating plate to 195° C until completely dissolved, then sequentially added with 3 g of sodium alginate (slowly in 5 parts) and 2 g of Na—CS porous activated zeolite from step (2), continuously stirred with a glass rod for 20 minutes to obtain a Na—CS zeolite-sodium alginate/polyvinyl alcohol mixture.
(4) The Na—CS zeolite-sodium alginate/polyvinyl alcohol mixture from step (3) was sterilized in an autoclave with 121° C steam for 20 minutes, and then cooled to room temperature. 50 ml of a Chlorella algae solution was centrifuged at 8000 r/min for 10 minutes. The obtained Chlorella sediment was added to the sterilized mixture and stirred continuously at 300 r/min for 30 minutes to obtain a uniformly dispersed algae mixture.
(5) 15 g of calcium chloride was added to 500 ml of saturated boric acid solution, stirred until calcium chloride was completely dissolved, and then sodium bicarbonate was used to adjust the pH value of the solution to 6.6 to obtain the cross-linking agent. The algae mixture from step (4) was dropped into the prepared cross-linking agent drop by drop using a 5-ml syringe to form immobilized algae beads, which were then transferred to a refrigerator and stored at 4° C for 24 hours to achieve low-temperature immobilization cross-linking. The cross-linking agent was discarded, and the immobilized algae beads were washed three times with deionized water to obtain modified-zeolite immobilized algae beads. The obtained immobilized algae beads have a diameter of 4.7 mm and a density of 1.09 g/cm³.

### Example 2

(1) The zeolite raw material was washed with deionized water three times, dried in a 60° C oven for 240 minutes. Then the natural zeolite was added to 0.8 mol/L sodium bicarbonate solution at a mass to volume ratio (g/L) of 15:1, stirred at 200 r/min for 12 hours, filtered, and dried. The dried zeolite was heated in a muffle furnace at a heating rate of 6° C/min to 350° C and heated at this temperature for 180 minutes, to obtain Na-type porous activated zeolite.
(2) Chitosan was dissolved in water, stirred at 30° C for 5 minutes, then added with glacial acetic acid and mixed evenly to prepare a mixed solution including 0.5% chitosan and 2% glacial acetic acid. 4 g of the Na-type porous activated zeolite prepared in step (1) was added to 100 mL of the mixed solution, vibrated and stirred at 140 r/min for 24 hours, then washed with deionized water until the filtrate is neutral, dried at 60° C for 120 minutes to obtain Na—CS porous activated zeolite. The obtained Na—CS porous activated zeolite was ground and sieved through a 300-mesh screen to obtain uniformly sized Na—CS porous activated zeolite.
(3) 6 g of polyvinyl alcohol 124 was added to 100 ml of water, heated on an electric heating plate to 195° C until completely dissolved, then sequentially added with 2 g of sodium alginate (slowly in 5 parts) and 4 g of Na—CS porous activated zeolite from step (2), continuously stirred with a glass rod for 20 minutes to obtain a Na—CS zeolite-sodium alginate/polyvinyl alcohol mixture.
(4) The Na—CS zeolite-sodium alginate/polyvinyl alcohol mixture from step (3) was sterilized in an autoclave with 121° C steam for 20 minutes, and then cooled to room temperature. 80 ml of a Chlorella algae solution was centrifuged at 8000 r/min for 10 minutes. The obtained Chlorella sediment was added to the sterilized mixture and stirred continuously at 300 r/min for 30 minutes to obtain a uniformly dispersed algae mixture.
(5) 20 g of calcium chloride was added to 500 ml of saturated boric acid solution, stirred until calcium chloride was completely dissolved, and then sodium bicarbonate was used to adjust the pH value of the solution to 6.6 to obtain the cross-linking agent. The algae mixture from step (4) was dropped into the prepared cross-linking agent drop by drop using a 5-ml syringe to form immobilized algae beads, which were then transferred to a refrigerator and stored at 4° C for 24 hours to achieve low-temperature immobilization cross-linking. The cross-linking agent was discarded, and the immobilized algae beads were washed three times with deionized water to obtain modified-zeolite immobilized algae beads. The obtained immobilized algae beads have a diameter of 5.1 mm and a density of 1.14 g/cm³.

### Example 3

(1) The zeolite raw material was washed with deionized water three times, dried in a 60° C oven for 240 minutes. Then the natural zeolite was added to 1.0 mol/L sodium bicarbonate solution at a mass to volume ratio (g/L) of 20:1, stirred at 200 r/min for 12 hours, filtered, and dried. The dried zeolite was heated in a muffle furnace at a heating rate of 6° C/min to 400° C and heated at this temperature for 240 minutes, to obtain Na-type porous activated zeolite.
(2) Chitosan was dissolved in water, stirred at 30° C for 5 minutes, then added with glacial acetic acid and mixed evenly to prepare a mixed solution including 1.0% chitosan and 4% glacial acetic acid. 5 g of the Na-type porous activated zeolite prepared in step (1) was added to 100 mL of the mixed solution, vibrated and stirred at 140 r/min for 24 hours, then washed with deionized water until the filtrate is neutral, dried at 60° C for 120 minutes to obtain Na—CS porous activated zeolite. The obtained Na—CS porous activated zeolite was ground and sieved through a 300-mesh screen to obtain uniformly sized Na—CS porous activated zeolite.
(3) 8 g of polyvinyl alcohol 124 was added to 100 ml of water, heated on an electric heating plate to 195° C until completely dissolved, then sequentially added with 2 g of sodium alginate (slowly in 5 parts) and 5 g of Na—CS porous activated zeolite from step (2), continuously stirred with a glass rod for 20 minutes to obtain a Na—CS zeolite-sodium alginate/polyvinyl alcohol mixture.
(4) The Na—CS zeolite-sodium alginate/polyvinyl alcohol mixture from step (3) was sterilized in an autoclave with 121° C steam for 20 minutes, and then cooled to room temperature. 100 ml of a Chlorella algae solution was centrifuged at 8000 r/min for 10 minutes. The obtained Chlorella sediment was added to the sterilized mixture and stirred continuously at 300 r/min for 30 minutes to obtain a uniformly dispersed algae mixture.
(5) 10 g of calcium chloride was added to 500 ml of saturated boric acid solution, stirred until calcium chloride was completely dissolved, and then sodium bicarbonate was used to adjust the pH value of the solution to 6.6 to obtain the cross-linking agent. The algae mixture from step (4) was dropped into the prepared cross-linking agent drop by drop using a 5-ml syringe to form immobilized algae beads, which were then transferred to a refrigerator and stored at 4° C for 24 hours to achieve low-temperature immobilization cross-linking. The cross-linking agent was discarded, and the immobilized algae beads were washed three times with deionized water to obtain modified-zeolite immobilized algae beads. The obtained immobilized algae beads have a diameter of 5.4 mm and a density of 1.21 g/cm³.

### Example 4

(1) The zeolite raw material was washed with deionized water three times, dried in a 60° C oven for 240 minutes. Then the natural zeolite was added to 0.8 mol/L sodium bicarbonate solution at a mass to volume ratio (g/L) of 25:1, stirred at 200 r/min for 12 hours, filtered, and dried. The dried zeolite was heated in a muffle furnace at a heating rate of 6° C/min to 400° C and heated at this temperature for 240 minutes, to obtain Na-type porous activated zeolite.
(2) Chitosan was dissolved in water, stirred at 30° C for 5 minutes, then added with glacial acetic acid and mixed evenly to prepare a mixed solution including 0.7% chitosan and 6% glacial acetic acid. 6 g of the Na-type porous activated zeolite prepared in step (1) was added to 100 mL of the mixed solution, vibrated and stirred at 140 r/min for 24 hours, then washed with deionized water until the filtrate is neutral, dried at 60° C for 120 minutes to obtain Na—CS porous activated zeolite. The obtained Na—CS porous activated zeolite was ground and sieved through a 300-mesh screen to obtain uniformly sized Na—CS porous activated zeolite.
(3) 4 g of polyvinyl alcohol 124 was added to 100 ml of water, heated on an electric heating plate to 195° C until completely dissolved, then sequentially added with 4 g of sodium alginate (slowly in 5 parts) and 6 g of Na—CS porous activated zeolite from step (2), continuously stirred with a glass rod for 20 minutes to obtain a Na—CS zeolite-sodium alginate/polyvinyl alcohol mixture.
(4) The Na—CS zeolite-sodium alginate/polyvinyl alcohol mixture from step (3) was sterilized in an autoclave with 121° C steam for 20 minutes, and then cooled to room temperature. 110 ml of a Chlorella algae solution was centrifuged at 8000 r/min for 10 minutes. The obtained Chlorella sediment was added to the sterilized mixture and stirred continuously at 300 r/min for 30 minutes to obtain a uniformly dispersed algae mixture.
(5) 10 g of calcium chloride was added to 500 ml of saturated boric acid solution, stirred until calcium chloride was completely dissolved, and then sodium bicarbonate was used to adjust the pH value of the solution to 6.6 to obtain the cross-linking agent. The algae mixture from step (4) was dropped into the prepared cross-linking agent drop by drop using a 5-ml syringe to form immobilized algae beads, which were then transferred to a refrigerator and stored at 4° C for 24 hours to achieve low-temperature immobilization cross-linking. The cross-linking agent was discarded, and the immobilized algae beads were washed three times with deionized water to obtain modified-zeolite immobilized algae beads. The obtained immobilized algae beads have a diameter of 4.5 mm and a density of 1.17 g/cm³.

### Example 5

(1) The zeolite raw material was washed with deionized water three times, dried in a 60° C oven for 240 minutes. Then the natural zeolite was added to 0.9 mol/L sodium bicarbonate solution at a mass to volume ratio (g/L) of 30:1, stirred at 200 r/min for 12 hours, filtered, and dried. The dried zeolite was heated in a muffle furnace at a heating rate of 5° C/min to 450° C and heated at this temperature for 120 minutes, to obtain Na-type porous activated zeolite.
(2) Chitosan was dissolved in water, stirred at 30° C for 5 minutes, then added with glacial acetic acid and mixed evenly to prepare a mixed solution including 1.5% chitosan and 4% glacial acetic acid. 4 g of the Na-type porous activated zeolite prepared in step (1) was added to 100 mL of the mixed solution, vibrated and stirred at 140 r/min for 24 hours, then washed with deionized water until the filtrate is neutral, dried at 60° C for 120 minutes to obtain Na—CS porous activated zeolite. The obtained Na—CS porous activated zeolite was ground and sieved through a 300-mesh screen to obtain uniformly sized Na—CS porous activated zeolite.
(3) 4 g of polyvinyl alcohol 124 was added to 100 ml of water, heated on an electric heating plate to 195° C until completely dissolved, then sequentially added with 2 g of sodium alginate (slowly in 5 parts) and 4 g of Na—CS porous activated zeolite from step (2), continuously stirred with a glass rod for 20 minutes to obtain a Na—CS zeolite-sodium alginate/polyvinyl alcohol mixture.
(4) The Na—CS zeolite-sodium alginate/polyvinyl alcohol mixture from step (3) was sterilized in an autoclave with 121° C steam for 20 minutes, and then cooled to room temperature. 120 ml of a Chlorella algae solution was centrifuged at 8000 r/min for 10 minutes. The obtained Chlorella sediment was added to the sterilized mixture and stirred continuously at 300 r/min for 30 minutes to obtain a uniformly dispersed algae mixture.
(5) 20 g of calcium chloride was added to 500 ml of saturated boric acid solution, stirred until calcium chloride was completely dissolved, and then sodium bicarbonate was used to adjust the pH value of the solution to 6.6 to obtain the cross-linking agent. The algae mixture from step (4) was dropped into the prepared cross-linking agent drop by drop using a 5-ml syringe to form immobilized algae beads, which were then transferred to a refrigerator and stored at 4° C for 24 hours to achieve low-temperature immobilization cross-linking. The cross-linking agent was discarded, and the immobilized algae beads were washed three times with deionized water to obtain modified-zeolite immobilized algae beads. The obtained immobilized algae beads have a diameter of 4.2 mm and a density of 1.12 g/cm³.

### Comparative Example 1

(1) The zeolite raw material was washed with deionized water three times, dried in a 60° C oven for 240 minutes. The dried zeolite was grounded and sieved through a 300-mesh screen to obtain uniformly sized natural zeolite.
(2) 2 g of sodium alginate (slowly in 5 parts) and 5 g of zeolite from step (1) were added to 100 ml of water, continuously stirred with a glass rod for 20 minutes to obtain a zeolite-sodium alginate mixture.
(3) The zeolite-sodium alginate mixture from step (2) was sterilized in an autoclave with 121° C steam for 20 minutes, and then cooled to room temperature. 100 ml of a Chlorella algae solution was centrifuged at 8000 r/min for 10 minutes. The obtained Chlorella sediment was added to the sterilized mixture and stirred continuously at 300 r/min for 30 minutes to obtain a uniformly dispersed algae mixture.
(4) 10 g of calcium chloride was added to 500 ml water, stirred until calcium chloride was completely dissolved, and then sodium bicarbonate was used to adjust the pH value of the solution to 6.6 to obtain the cross-linking agent. The algae mixture from step (3) was dropped into the prepared cross-linking agent drop by drop using a 5-ml syringe to form immobilized algae beads, which were then transferred to a refrigerator and stored at 4° C for 24 hours to achieve low-temperature immobilization cross-linking. The cross-linking agent was discarded, and the immobilized algae beads were washed three times with deionized water to obtain immobilized algae beads. The obtained immobilized algae beads have a diameter of 3.9 mm and a density of 1.06 g/cm³.

### Comparative Example 2

(1) The zeolite raw material was washed with deionized water three times, dried in a 60° C oven for 240 minutes. Then the natural zeolite was added to 0.7 mol/L sodium bicarbonate solution at a mass to volume ratio (g/L) of 10:1, stirred at 200 r/min for 12 hours, filtered, and dried. The dried zeolite was heated in a muffle furnace at a heating rate of 4° C/min to 300° C and heated at this temperature for 120 minutes, to obtain Na-type porous activated zeolite.
(2) The obtained Na-type porous activated zeolite was ground and sieved through a 300-mesh screen to obtain uniformly sized Na-type porous activated zeolite.
(3) 4 g of polyvinyl alcohol 124, whose degree of alcoholysis was 99%, average degree of polymerization was 2500, and viscosity was 56 mPa·s, was added to 100 ml of water, heated on an electric heating plate to 195° C until completely dissolved, then sequentially added with 3 g of sodium alginate (slowly in 5 parts) and 2 g of Na-type porous activated zeolite from step (2), continuously stirred with a glass rod for 20 minutes to obtain a Na-type zeolite-sodium alginate/polyvinyl alcohol mixture.
(4) The Na-type zeolite-sodium alginate/polyvinyl alcohol mixture from step (3) was sterilized in an autoclave with 121° C steam for 20 minutes, and then cooled to room temperature. 50 ml of a Chlorella algae solution was centrifuged at 8000 r/min for 10 minutes. The obtained Chlorella sediment was added to the sterilized mixture and stirred continuously at 300 r/min for 30 minutes to obtain a uniformly dispersed algae mixture.
(5) 15 g of calcium chloride was added to 500 ml of saturated boric acid solution, stirred until calcium chloride was completely dissolved, and then sodium bicarbonate was used to adjust the pH value of the solution to 6.6 to obtain the cross-linking agent. The algae mixture from step (4) was dropped into the prepared cross-linking agent drop by drop using a 5-ml syringe to form immobilized algae beads, which were then transferred to a refrigerator and stored at 4° C for 24 hours to achieve low-temperature immobilization cross-linking. The cross-linking agent was discarded, and the immobilized algae beads were washed three times with deionized water to obtain modified-zeolite immobilized algae beads. The obtained immobilized algae beads have a diameter of 4.5 mm and a density of 1.08 g/cm³.

The immobilized algae beads formed in Examples 1 to 5 and Comparative Examples 1 to 2 were each introduced into raw water samples with the same water quality at a concentration of 15 g/L for adsorption performance test. After 2 days, the concentrations of NH₄⁺—N, NO₃⁻—N, TP, TN, and COD in the treated water and the leakage amount of algae cells were measured. The methods for measuring the concentrations of NH₄⁺—N, NO₃⁻—N, TP, TN, and COD are shown in Table 1, and the test results are shown in Table 2.

The values of indicators of the raw water in the adsorption performance test were as follows: NH₄⁺—N at 15.77 mg/L, NO₃⁻—N at 35.13 mg/L, TP at 2.41 mg/L, TN at 50.9 mg/L, and COD at 64 mg/L.

TABLE 1

| Tested Indicator | Test Method |
|---|---|
| NH₄⁺—N | Nessler's Reagent Spectrophotometry Method |
| NO₃⁻—N | Ammonium Molybdate Spectrophotometry Method |
| TP | Potassium Persulfate Digestion Ultraviolet |
| | Spectrophotometry Method |
| TN | Alkaline Potassium Persulfate Digestion |
| | Ultraviolet Spectrophotometry |
| COD | Potassium Dichromate HACH Digestion Method |

The method for measuring the leakage amount of algae cells was as follows: The biomass of Chlorella was determined by measuring the dry weight of the algae solution. Since directly measuring the dry weight of the algae solution is not convenient for dynamically monitoring the biomass of algae cells, the absorbance of the algae solution at a wavelength of 680 nm is converted into the dry weight of the algae solution to reflect the biomass. The absorbance of Chlorella algae solutions of various concentrations at 680 nm wavelength was measured using a UV-visible spectrophotometer. 0.45 μm fiber filter membranes were dried until reaching constant weights. The Chlorella algae solutions of various concentrations were respectively filtered through the dried filter membranes and dried at 105° C until reaching constant weights, cooled to room temperature, and then weighed, thereby determining the dry weights of algae cells in the solutions. A standard curve correlating the absorbance of the Chlorella algae solution with biomass was established:

Y=0.4361·x−0.00642(R²=0.998)

In this formula, x represents the OD680 (optical density at 680 nm) of the Chlorella algae solution, Y represents the biomass of Chlorella (g/L).

TABLE 2

| | Example | Example | Example | Example | Example | Comparative | Comparative |
|---|---|---|---|---|---|---|---|
| Group | 1 | 2 | 3 | 4 | 5 | Example 1 | Example 2 |
| Raw water | 15.77 | 15.77 | 15.77 | 15.77 | 15.77 | 15.77 | 15.77 |
| NH₄⁺—N (mg/L) | | | | | | | |
| Treated Water | 3.84 | 2.13 | 1.06 | 1.79 | 2.92 | 7.79 | 5.75 |
| NH₄⁺—N (mg/L) | | | | | | | |
| NH₄⁺—N | 75.65 | 86.49 | 93.28 | 88.65 | 81.48 | 50.60 | 63.54 |
| Removal Rate | | | | | | | |
| Raw water | 35.13 | 35.13 | 35.13 | 35.13 | 35.13 | 35.13 | 35.13 |
| NO₃⁻—N (mg/L) | | | | | | | |
| Treated Water | 15.75 | 12.28 | 7.96 | 10.15 | 9.44 | 20.18 | 18.59 |
| NO₃⁻—N (mg/L) | | | | | | | |
| NO₃⁻—N | 55.17 | 65.04 | 77.34 | 71.11 | 73.13 | 42.56 | 47.08 |
| Removal Rate | | | | | | | |
| (%) | | | | | | | |
| Raw water | 2.41 | 2.41 | 2.41 | 2.41 | 2.41 | 2.41 | 2.41 |
| TP(mg/L) | | | | | | | |
| Treated Water | 0.94 | 0.74 | 0.31 | 0.55 | 0.55 | 1.34 | 1.06 |
| TP(mg/L) | | | | | | | |
| TP Removal | 61.00 | 69.29 | 87.14 | 77.18 | 77.18 | 44.40 | 56.02 |
| Rate (%) | | | | | | | |
| Raw water | 50.9 | 50.9 | 50.9 | 50.9 | 50.9 | 50.9 | 50.9 |
| TN(mg/L) | | | | | | | |
| Treated Water | 20.97 | 15.61 | 9.88 | 12.33 | 13.94 | 29.81 | 25.23 |
| TN(mg/L) | | | | | | | |
| TN Removal | 58.80 | 69.33 | 80.59 | 75.78 | 72.61 | 41.43 | 50.43 |
| Rate (%) | | | | | | | |
| Raw water | 64 | 64 | 64 | 64 | 64 | 64 | 64 |
| COD(mg/L) | | | | | | | |
| Treated Water | 25 | 18 | 13 | 13 | 14 | 36 | 30 |
| COD(mg/L) | | | | | | | |
| COD Removal | 60.94 | 71.88 | 79.69 | 79.69 | 78.13 | 43.75 | 53.13 |
| Rate (%) | | | | | | | |
| Leakage amount | 13.5 | 9.1 | 3.2 | 7.8 | 11.0 | 23.7 | 16.2 |
| of algae cells | | | | | | | |
| (mg/L) | | | | | | | |

It can be seen from the above table that, compared to the conventional sodium alginate algae beads in Comparative Example 1 and the immobilized algae beads formed with zeolite that is not modified with chitosan in Comparative Example 2, the modified-zeolite immobilized algae beads formed in Examples 1 to 5 of the present application exhibited significantly higher removal rates for NH₄⁺—N, NO₃⁻—N, TP, and COD in water, as well as significantly reduced algae cell leakage. Particularly, the modified-zeolite immobilized algae beads formed in Example 3 exhibited increase in the removal rates of NH₄⁺—N, NO₃⁻—N, TP, TN, and COD compared to Comparative Example 1 by 42.68%, 34.78%, 42.74%, 39.16%, and 35.94%, respectively, and the algae cell leakage was reduced by 20.5 mg/L compared to the conventional sodium alginate algae beads, further proving that the modified-zeolite immobilized algae beads provided in the present application possess excellent adsorption performance. These immobilized algae beads not only possess improved mechanical strength, embedding performance, and service life but also reduce the operational difficulty and cost for separating Chlorella and zeolite from wastewater, thereby offering great potential for practical applications.

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the present application.

The above-described embodiments are only several implementations of the present application, and the descriptions are relatively specific and detailed, but they should not be construed as limiting the scope of the present application. It should be understood by those of ordinary skill in the art that various modifications and improvements can be made without departing from the concept of the present application, and all fall within the protection scope of the present application. Therefore, the patent protection of the present application shall be defined by the appended claims.

1. A method for preparing modified-zeolite immobilized algae beads, the method comprising:
obtaining porous zeolite;
mixing the porous zeolite with a mixed solution comprising chitosan, glacial acetic acid, and water to prepare chitosan-modified porous zeolite; and
mixing the chitosan-modified porous zeolite with an embedding agent, Chlorella, and a cross-linking agent to prepare the modified-zeolite immobilized algae beads.

2. The method of claim 1, wherein
a deacetylation degree the of the chitosan is greater than or equal to 95%.

3. The method of claim 1, wherein
a mass fraction of chitosan in the mixed solution is ranged from 0.4% to 1.5%.

4. The method of claim 1, wherein the embedding agent comprises polyvinyl alcohol and sodium alginate.

5. The method of any-m 1, wherein the cross-linking agent comprises a saturated boric acid solution and calcium chloride.

6. The method of claim 4, wherein a mass ratio of polyvinyl alcohol to sodium alginate is ranged from (4 to 8):(2 to 4); and a mass ratio of polyvinyl alcohol to the chitosan-modified porous zeolite is ranged from (4 to 8):(2 to 6).

7. The method of claim 5, wherein a mass to volume ratio of calcium chloride to the saturated boric acid solution is ranged from (10 to 20) g:500 mL.

8. The method of claim 1, wherein the porous zeolite comprises Na-type porous zeolite.

9. The method of claim 8, wherein a preparation method for the Na-type porous zeolite comprises:
adding zeolite to a sodium bicarbonate solution, and heating at a temperature ranged from 300° C to 450° C, thereby forming the Na-type porous activated zeolite.

10. The method of claim 9, wherein
the zeolite comprises one or more of clinoptilolite, mordenite, heulandite, or chabazite.

11-15. (canceled)

16. The method of claim 1, wherein a viscosity of the chitosan is ranged from 100 mPa·s to 200 mPa·s.

17. The method of claim 1, wherein a mass fraction of glacial acetic acid in the mixed solution is ranged from 2% to 6%.

18. The method of claim 1, wherein a mass to volume ratio of porous zeolite to the mixed solution is ranged from (2 to 6) g:100 mL.

19. The method of claim 9, wherein a concentration of the sodium bicarbonate solution is ranged from 0.7 mol/L to 1.0 mol/L.

20. The method of claim 9, wherein a mass to volume ratio of the zeolite to the sodium bicarbonate solution is ranged from (10 to 30) g:1 L.

21. The method of claim 9, wherein a duration of heating is ranged from 120 min to 240 min.

22. Modified-zeolite immobilized algae beads prepared by the method of claim 1.

23. The modified-zeolite immobilized algae beads of claim 22, wherein a diameter of the modified-zeolite immobilized algae beads is ranged from 4.2 mm to 5.4 mm.

24. The modified-zeolite immobilized algae beads of claim 22, wherein a density of the modified-zeolite immobilized algae beads is ranged from 1.09 g/cm³ to 1.21 g/cm³.

25. A wastewater treating method, comprising introducing the modified-zeolite immobilized algae beads of claim 22 at a ratio ranged from 10 g/L to 20 g/L into wastewater for water treatment, wherein a C/N ratio of the wastewater is ranged from 1 to 3.
